(19) United States
(12) Patent Application Publication
(10) Pub. No.: US 2020/0261373 A1
(43) Pub. Date: Aug. 20, 2020

(54) PHARMACEUTICAL CAPSULE CONTAINING AT LEAST TWO TABLETS

(71) Applicant: RECKITT BENCKISER LLC, Parsippany, NJ (US)

(72) Inventors: NILS WILLIAM AHLGREN, CRANBERRY TOWNSHIP, PA (US); RAGHU CAVATUR, PARSIPPANY, NJ (US); KEVIN CHEN, PARSIPPANY, NJ (US); MATTHEW JAMES KASER, PARSIPPANY, NJ (US); HONGCHUN QIU, Basking Ridge, NJ (US)

(21) Appl. No.: 16/866,136

(22) Filed: May 4, 2020

Related U.S. Application Data
(63) Continuation of application No. 15/520,731, filed on Apr. 20, 2017, now abandoned.
(60) Provisional application No. 62/066,536, filed on Oct. 21, 2014.

(51) Int. Cl. A61K 9/48; A61K 45/06; A61K 9/20; A61K 31/485; A61K 31/192; A61K 31/09

(52) U.S. Cl. CPC A61K 9/4808; A61K 45/06; A61K 9/2059; A61K 9/2027; A61K 9/2054; A61K 9/2013; A61K 31/485; A61K 31/192; A61K 31/09; A61K 9/2018

(57) ABSTRACT

The present invention is directed to an oral pharmaceutical dosage form comprising a capsule containing at least two tablets, each tablet containing at least one different pharmaceutically active ingredient

The present invention relates to a novel pharmaceutical dosage form and in particular to a capsule for oral administration to human subjects which contains at least two different pharmaceutical compositions each containing at least one active pharmaceutical ingredient. Typically, the capsule is in the form of a gelatin or cellulose based capsule.

Most solid dose medicines comprise a single active pharmaceutical ingredient. As a result, administration of several active pharmaceutical ingredients to provide relief to a patient from a number of different symptoms can be complex. If the active pharmaceutical ingredients have to be taken in different dosage units, the patient can encounter the challenge of ensuring that their medication is taken at the correct time and in a compliant way. Any compromise in following the product instructions on how to take the medication by the patient could have a significant negative impact on the therapeutic benefits and even safety of the drug product.

Fixed-dose combination products are defined as a drug product which contain more than one active pharmaceutical ingredient and known for solid oral dosage forms. Typically, these will be based on two pharmaceutical active ingredients in the same tablet or caplet. Alternative formats include the use of a gelatin capsule which has separate compartments each of which contains a composition comprising a different active. The use of a first gelatin capsule located in a second, larger gelatin capsule has also been proposed. However, these alternative dosage forms have not been commercialized—possibly due to difficulties encountered in terms of manufacturing, for example in handling, filling and closing the separate caps., returns for filling and closure of the compartments.

The use of microtablets or pellets has also been disclosed. However, the production of microtablets is not easy. The size of the microtablets is such that control of the excipients to achieve the desired release profile can be difficult—this is particularly true of sustained release tablets. In addition, on dissolution the swelling of the microtablets can be such that they stick together and there is the potential for the tablets to stick to the material of the capsule in which they are contained.

Capsules are often preferred by patients over compressed tablets because they are easier to swallow. Immediately upon contact with the moisture in the human mouth they can become exceedingly slippery and slide down the throat easily and without the friction on the mucus membrane associated with compressed tablets.

Capsules (usually gelatin capsules) that are generally available and incorporate two different pharmaceutical compositions such as, e.g., an immediate release composition and a sustained-release composition are made by very expensive methods of manufacture that require either fluid bed coaters or pharmaceutical manufacturing equipment that provides the compositions by extrusion and spheronization. These methods permit precise amounts of active pharmaceutical agents to be sprayed onto beads or to prepare pharmaceutical compositions in the form of pellets which are then put into the capsules.

A further reason why formulations having an immediate release matrix and a controlled release matrix are not widely used in capsules in the past is due to the fact that all formulations that comprise two or more of such different matrices have usually been compressed into tablets. As this is the traditional method of manufacturing pharmaceutical dosage forms using immediate and sustained release matrices, many of the inactive ingredients (excipients) commonly used in tablet formulations are too bulky—unless compressed by powerful tablet presses—to fit the required and/or desired quantity of the formulations into capsules of a size that is small enough to be suitable for oral ingestion by humans.

According to a first aspect of the present invention there is provided an oral pharmaceutical dosage form comprising a capsule containing at least two tablets, each tablet containing at least one different pharmaceutically active ingredient wherein each of the at least two tablets have different dimensions.

The capsule can be made of any suitable material, such as gelatin, alginate, or cellulose including hydroxylpropyl methyl cellulose. In a preferred embodiment the capsule is a soft/hard gelatin capsule. In a preferred embodiment, the capsule is obtained from two shells of hard gelatin which are sealed together around the combined medicaments. Alternatively, the capsule can be a one-piece capsule. Soft gelatin capsules are usually prepared from gelatin, glycerin and water, and can absorb several times their own weight in water. Other non-limiting materials for making capsules of the present invention include cellulose esters and/or ethers such as, e.g., hydroxypropylmethylcellulose (HPMC).

Typically, the at least two tablets are not in the form of a powdered or granulated composition.

Preferably the at least two tablets do not comprise a natural gum, such as xanthan gum.

After dissolution of the capsule the at least two tablets release the active pharmaceutical ingredients contained therein comparably to each of the at least two tablets being administered individually.

The oral pharmaceutical dosage form can comprise two, three or four different compositions.

The at least two tablets can have the same or a different geometric form, the same or a different weight, and the same or a different volume with the proviso that at least one of the geometric form, weight or volume is different between the tablets.

For the avoidance of doubt, in the event that the dosage form comprises three or more tablets then two or more of the tablets can have the same dimensions.

The at least two tablets can be coated or uncoated.

The dimensions of the capsule can be selected such that the pharmaceutical dosage form has shape which is compatible with easy swallowing. The capsule can generally be in the shape of a sphere or an elongated sphere (oblong form).

The capsule can have a length of up to 35 mm, a width of up to 15 mm and a depth of up to 15 mm. In a preferred embodiment the capsule can have a length of 25-30 mm, a width of 8-10, and a depth of 8-10 mm. In a preferred embodiment the capsule can have a length of 20-24 mm, a width of 8-10, and a depth of 8-10 mm.

The capsule can have a volume of 0.75 ml-1.5 ml. A preferred volume can be 0.90-1.05 ml.

The at least two tablets can have a surface that is complementary to the face of an adjacent tablet, the two faces being intended to be opposite one another in the final pharmaceutical dosage form. The two faces can be planar or substantially planar. Alternatively, the at least two tablets can have a round or ovoid/oval geometry.

The geometric form of the at least two tablets is adapted to the needs of the final dosage form. For example, an existing tablet can be used but its dimensions can be altered without changing the qualitative and quantitative composition of the original tablet.

The invention relates also to oral pharmaceutical combinations which are presented in the pharmaceutical dosage form according to the invention and the purpose of which is to provide a therapeutic treatment by joint administration of pharmaceutical active ingredients (especially three pharmaceutical active ingredients), in which the oral pharmaceutical dosage form according to the invention is administered to a patient who needs it.

The at least two different tablets may comprise one or more active pharmaceutical active ingredients selected from analgesics, antihistamines, decongestants, antitussives, expectorants, vitamins, probiotics, anti-inflammatories, anti-infectives, antibiotics, acid reducers, and laxatives.

In a preferred embodiment the at least two tablets comprise an analgesic, an expectorant and at least one additional active pharmaceutical ingredient.

The analgesic can be selected from naproxen, ketoprofen, diclofenac, ibuprofen, paracetamol, aspirin and flurbiprofen

The expectorant can be guaifenesin or N-acetyl cysteine (NAC).

The at least one other drug can be selected from an antitussive or cough suppressant such as dextromethorphan, dextromethorphan hydrobromide, codeine, codeine phosphate, codeine sulphate, diphenhydramine citrate, diphenhydramine hydrochloride and diphenhydramine citrate, a decongestant such as phenylephrine hydrochloride, phenylpropanolamine hydrochloride, pseudoephedrine hydrochloride or ephedrine, an antihistamine such as chlorpheniramine maleate, brompheniramine maleate, phenindamine tartrate, pyrilamine maleate, doxylamine succinate, phenyltoloxamine citrate, diphenhydramine hydrochloride, diphenhydramine citrate, promethazine, and clemastine fumerate, or a combination thereof. Preferred actives are dextromethorphan hydrobromide or pseudoephedrine hydrochloride.

The invention relates also to the use of a pharmaceutical dosage form as described in the first aspect comprising an analgesic, an expectorant and another active such as a decongestant for relief from the symptoms of cough, cold or flu in a patient.

According to another aspect of the present invention there is provided the use of a pharmaceutical dosage form as described in the first aspect of the present invention for the treatment of a patient wherein a single dose provides the required therapeutic effect for a period of up to 12 hours.

According to another aspect of the present invention there is provided the use of a pharmaceutical dosage form as described in the first aspect of the present invention for the treatment of a patient wherein a single dose provides the required therapeutic effect for a period of up to 12 hours for the treatment of and/or relief from the symptoms of cough, cold or flu.

The at least two tablets are designed such that the pharmaceutical active ingredients have coordinated therapeutic dosages. As a result, the dosages of the tablets are optimised according to an optimum therapeutic effectiveness in a single administration. Typically, the at least two tablets provide a therapeutic effect for a period of up to 12 hrs, irrespective of their different elimination half-lives of the active ingredients contained wherein. This is often time highly difficult to achieve with other solid oral dosage forms such as tablet, especially with a fixed-dose combination products which contains more than two pharmaceutical active ingredients.

According to a yet further aspect of the present invention there is provided a process for producing oral pharmaceutical dosage form as described in the first aspect of the present invention. The process comprises a step of bringing the at least two tablets together in a capsule.

The capsule of the present invention may comprise at least two compositions which differ with respect to at least the release profiles thereof. For example, the at least two different pharmaceutical compositions may comprise the same pharmaceutical active ingredients and provide two different release profiles of the pharmaceutical active ingredient such as, e.g., an immediate release composition and a controlled release composition.

The at least two different tablets may comprise at least two different pharmaceutical active ingredients. Further, one of the at least two compositions may be an immediate release composition and the other one may be a controlled release composition.

The present invention also provides a gelatin or HPMC capsule for oral administration to a human subject which comprises two different tablets which comprise a mixture of one or more active pharmaceutical ingredients and one or more excipients. One of the tablets is an immediate release tablet and comprises at least one first active pharmaceutical ingredient. The second tablet comprises both an immediate release portion and a sustained release portion and comprises at least one second active pharmaceutical ingredient which is the same as or different from the first active pharmaceutical ingredient. Typically, the second tablet comprises a different active pharmaceutical ingredient(s) from the first tablet. The capsule comprises a therapeutically effective amount of the or each active ingredient contained therein.

As set forth herein, “portion” means a part of a whole, either separated or integrated with it. Thus, a product with two or more portions may have, but does not necessarily require, separate or discrete structural elements. As further set forth herein “sustained release” refers to a type of “modified release”, and these terms are used interchangeably throughout.

In a preferred embodiment, the first tablet is an immediate release tablet which comprises naproxen. The second tablet comprises an immediate release portion comprising guaifenesin and dextromethorphan and a sustained release portion comprising guaifenesin and dextromethorphan.

In an embodiment the first tablet can comprise:
(a) 50-52% Naproxen;
(b) 35-40% Microcrystalline cellulose;
(c) up to 5% Povidone;
(d) 5-8% Croscarmellose sodium; and
(e) up to 1% Magnesium stearate.

In an alternative embodiment the first tablet can comprise:
(a) 70-75% Naproxen;
(b) 20-25% Microcrystalline cellulose;
(c) up to 5% Povidone;
(d) up to 1% Magnesium stearate.

In a further alternative embodiment the first tablet can comprise:
(a) 70-75% Naproxen;
(b) 9-10% Microcrystalline cellulose;
(c) up to 5% Povidone;
(d) up to 1% Magnesium stearate;
(e) 5-10% Lactose; and
(f) 5-10% Croscarmellose sodium.

In a yet further alternative embodiment the first tablet can comprise:
(a) 70-75% Naproxen;
(b) 6-7% Microcrystalline cellulose;
(c) up to 5% Povidone;
(d) up to 1% Magnesium stearate;
(e) 1-2% Croscarmellose sodium; and
(f) 15-20% Sodium lauryl sulfate.

In a yet further alternative embodiment the first tablet can comprise:
(a) 70-75% Naproxen;
(b) 3-5% Microcrystalline cellulose;
(c) up to 5% Povidone;
(d) up to 1% Magnesium stearate;
(e) up to 5% Lactose; and
(f) 15-20% Sodium lauryl sulfate.

In a yet further alternative embodiment the first tablet can comprise:
(a) 70-75% Naproxen;
(b) 9-10% Microcrystalline cellulose;
(c) up to 5% Povidone;
(d) up to 1% Magnesium stearate;
(e) up to 5% Croscarmellose sodium; and
(f) 5-10% Lactose.

In a yet further alternative embodiment the first tablet can comprise:
(a) 70-75% Naproxen or a pharmaceutically acceptable salt thereof;
(b) 5-10% Microcrystalline cellulose;
(c) up to 5% Povidone;
(d) up to 1% Magnesium stearate;
(e) 5-10% Lactose; and
(f) up to 5% Croscarmellose sodium.

In a yet further alternative embodiment the first tablet can comprise:
(a) 70-75% Naproxen or a pharmaceutically acceptable salt thereof;
(b) 5-10% Microcrystalline cellulose;
(c) up to 5% Povidone;
(d) up to 1% Magnesium stearate;
(e) 5-10% Lactose; and
(f) 5-10% Croscarmellose sodium.

In a yet further alternative embodiment the first tablet can comprise:
(a) 70-75% Naproxen or a pharmaceutically acceptable salt thereof;
(b) 5-10% Microcrystalline cellulose;
(c) up to 5% Povidone;
(d) up to 1% Magnesium stearate;
(e) up to 5% Lactose; and
(f) 15-20% Sodium Lauryl Sulphate.

In a yet further alternative embodiment the first tablet can comprise:
(a) 70-75% Naproxen or a pharmaceutically acceptable salt thereof;
(b) 5-10% Microcrystalline cellulose;
(c) up to 5% Povidone;
(d) up to 1% Magnesium stearate;
(e) up to 2% Croscarmellose sodium; and
(f) 15-20% Sodium Lauryl Sulphate.

In an embodiment the immediate release portion of the second tablet can comprise:
(a) 30-40% Guaifenesin;
(b) up to 5% Dextromethorphan;
(c) 50-60% Microcrystalline cellulose;
(d) up to 7% Povidone;
(e) up to 7% Croscarmellose sodium; and
(f) up to 1% Magnesium stearate.

In an alternative embodiment the immediate release portion of the second tablet can comprise:
(a) 40-50% Guaifenesin;
(b) up to 5% Dextromethorphan HBr;
(c) 40-50% Microcrystalline cellulose;
(d) up to 5% Hypromellose;
(e) up to 5% Sodium starch glycolate; and
(f) up to 1% Magnesium stearate.

In a further alternative embodiment the immediate release portion of the second tablet can comprise:
(a) 60-65% Guaifenesin;
(b) up to 5% Dextromethorphan HBr;
(c) 25-30% Microcrystalline cellulose;
(d) up to 5% Povidone;
(e) up to 5% Croscarmellose Sodium; and
(f) up to 1% Magnesium stearate.

In a further alternative embodiment the immediate release portion of the second tablet can comprise:
(a) 50-60% Guaifenesin;
(b) up to 5% Dextromethorphan HBr;
(c) 35-40% Microcrystalline cellulose;
(d) up to 5% Povidone;
(e) up to 5% Croscarmellose Sodium; and
(f) up to 1% Magnesium stearate.

In a further alternative embodiment the immediate release portion of the second tablet can comprise:
(a) 40-50% Guaifenesin;
(b) up to 5% Dextromethorphan or a pharmaceutically acceptable salt thereof;
(c) 40-50% Microcrystalline cellulose;
(d) up to 5% Hypromellose;
(e) up to 5% Sodium starch glycolate; and
(f) up to 1% Magnesium stearate.

In a further alternative embodiment the immediate release portion of the second tablet can comprise:
(a) 50-65% Guaifenesin;
(b) up to 5% Dextromethorphan or a pharmaceutically acceptable salt thereof;
(c) 25-40% Microcrystalline cellulose;
(d) up to 5% Povidone;
(e) up to 5% Croscarmellose sodium; and
(f) up to 1% Magnesium stearate.

In an embodiment of the second tablet the modified release portion can comprise:
(a) 80-90% Guaifenesin;
(b) up to 5% Dextromethorphan HBr;
(c) up to 10% Hypromellose;
(d) up to 5% Carbomer;
(e) up to 1.55% Magnesium stearate.

In an alternative embodiment of the second tablet the modified release portion can comprise:
(a) 80-90% Guaifenesin;
(b) 3-6% Dextromethorphan HBr;
(c) up to 5% Hypromellose;
(d) up to 2% Hydroxy ethylcellulose;
(e) up to 5% Microcrystalline cellulose;
(f) up to 1% Magnesium stearate.

In an alternative embodiment of the second tablet the modified release portion can comprise:
(a) 80-90% Guaifenesin;
(b) 3-6% Dextromethorphan HBr;
(c) up to 5% Hypromellose;
(d) up to 5% Hydroxyethylcellulose;
(e) up to 5% Microcrystalline cellulose; and
(f) up to 1% Magnesium stearate.

In an alternative embodiment of the second tablet the modified release portion can comprise:
(a) 80-90% Guaifenesin;
(b) 3-6% Dextromethorphan HBr;
(c) 5-10% Hypromellose;
(d) up to 5% Hydroxyethylcellulose;
(e) up to 5% Microcrystalline cellulose;
(f) up to 1% Magnesium stearate.

In an alternative embodiment of the second tablet the modified release portion can comprise:
(a) 85-90% Guaifenesin;
(b) 4-5% Dextromethorphan or a pharmaceutically acceptable salt thereof;
(c) 3-6% Hypromellose;
(d) 1-3% Carbomer;
(e) 0.5-1% Magnesium stearate.

In an alternative embodiment of the second tablet the modified immediate release portion can comprise:
(a) 90-93% Guaifenesin;
(b) 4-6% Dextromethorphan or a pharmaceutically acceptable salt thereof;
(c) 1-3% Carbomer;
(d) 0.5-1% Magnesium stearate.

In an embodiment the first tablet consists essentially of:
(a) 50-52% Naproxen;
(b) 35-40% Microcrystalline cellulose;
(c) up to 5% Povidone;
(d) 5-8% Croscarmellose sodium; and
(e) up to 1% Magnesium stearate.

In an alternative embodiment the first tablet consists essentially of:
(a) 70-75% Naproxen;
(b) 20-25% Microcrystalline cellulose;
(c) up to 5% Povidone;
(d) up to 1% Magnesium stearate.

In a further alternative embodiment the first tablet consists essentially of:
(a) 70-75% Naproxen;
(b) 9-10% Microcrystalline cellulose;
(c) up to 5% Povidone;
(d) up to 1% Magnesium stearate;
(e) 5-10% Lactose; and
(f) 5-10% Croscarmellose sodium.

In a yet further alternative embodiment the first tablet consists essentially of:
(a) 70-75% Naproxen;
(b) 6-7% Microcrystalline cellulose;
(c) up to 5% Povidone;
(d) up to 1% Magnesium stearate;
(e) 1-2% Croscarmellose sodium; and
(f) 15-20% Sodium lauryl sulfate.

In a yet further alternative embodiment the first tablet consists essentially of:
(a) 70-75% Naproxen;
(b) 3-5% Microcrystalline cellulose;
(c) up to 5% Povidone;
(d) up to 1% Magnesium stearate;
(e) up to 5% Lactose; and
(f) 15-20% Sodium lauryl sulfate.

In a yet further alternative embodiment the first tablet consists essentially of:
(a) 70-75% Naproxen;
(b) 9-10% Microcrystalline cellulose;
(c) up to 5% Povidone;
(d) up to 1% Magnesium stearate;
(e) up to 5% Croscarmellose sodium; and
(f) 5-10% Lactose.

In a yet further alternative embodiment the first tablet consists essentially of:
(a) 70-75% Naproxen or a pharmaceutically acceptable salt thereof;
(b) 5-10% Microcrystalline cellulose;
(c) up to 5% Povidone;
(d) up to 1% Magnesium stearate;
(e) 5-10% Lactose; and
(f) up to 5% Croscarmellose sodium.

In a yet further alternative embodiment the first tablet consists essentially of:
(a) 70-75% Naproxen or a pharmaceutically acceptable salt thereof;
(b) 5-10% Microcrystalline cellulose;
(c) up to 5% Povidone;
(d) up to 1% Magnesium stearate;
(e) 5-10% Lactose; and
(f) 5-10% Croscarmellose sodium.

In a yet further alternative embodiment the first tablet consists essentially of:
(a) 70-75% Naproxen or a pharmaceutically acceptable salt thereof;
(b) 5-10% Microcrystalline cellulose;
(c) up to 5% Povidone;
(d) up to 1% Magnesium stearate;
(e) up to 5% Lactose; and
(f) 15-20% Sodium Lauryl Sulphate.

In a yet further alternative embodiment the first tablet consists essentially of:
(a) 70-75% Naproxen or a pharmaceutically acceptable salt thereof;
(b) 5-10% Microcrystalline cellulose;
(c) up to 5% Povidone;
(d) up to 1% Magnesium stearate;
(e) up to 2% Croscarmellose sodium; and
(f) 15-20% Sodium Lauryl Sulphate.

In an embodiment the immediate release portion of the second tablet consists essentially of:
(a) 30-40% Guaifenesin;
(b) up to 5% Dextromethorphan;
(c) 50-60% Microcrystalline cellulose;
(d) up to 7% Povidone;
(e) up to 7% Croscarmellose sodium; and
(f) up to 1% Magnesium stearate.

In an alternative embodiment the immediate release portion of the second tablet consists essentially of:
(a) 40-50% Guaifenesin;
(b) up to 5% Dextromethorphan HBr;
(c) 40-50% Microcrystalline cellulose;
(d) up to 5% Hypromellose;
(e) up to 5% Sodium starch glycolate; and
(f) up to 1% Magnesium stearate.

In a further alternative embodiment the immediate release portion of the second tablet consists essentially of:
(a) 60-65% Guaifenesin;
(b) up to 5% Dextromethorphan HBr;
(c) 25-30% Microcrystalline cellulose;
(d) up to 5% Povidone;
(e) up to 5% Croscarmellose Sodium; and
(f) up to 1% Magnesium stearate.

In a further alternative embodiment the immediate release portion of the second tablet consists essentially of:
(a) 50-60% Guaifenesin;
(b) up to 5% Dextromethorphan HBr;
(c) 35-40% Microcrystalline cellulose;
(d) up to 5% Povidone;
(e) up to 5% Croscarmellose Sodium; and
(f) up to 1% Magnesium stearate.

In a further alternative embodiment the immediate release portion of the second tablet consists essentially of:
(a) 40-50% Guaifenesin;
(b) up to 5% Dextromethorphan or a pharmaceutically acceptable salt thereof;
(c) 40-50% Microcrystalline cellulose;
(d) up to 5% Hypromellose;
(e) up to 5% Sodium starch glycolate; and
(f) up to 1% Magnesium stearate.

In a further alternative embodiment the immediate release portion of the second tablet consists essentially of:
(a) 50-65% Guaifenesin;
(b) up to 5% Dextromethorphan or a pharmaceutically acceptable salt thereof;
(c) 25-40% Microcrystalline cellulose;
(d) up to 5% Povidone;
(e) up to 5% Croscarmellose sodium; and
(f) up to 1% Magnesium stearate.

In an embodiment of the second tablet the modified release portion consists essentially of:
(a) 80-90% Guaifenesin;
(b) up to 5% Dextromethorphan HBr;
(c) up to 10% Hypromellose;
(d) up to 5% Carbomer;
(e) up to 1.55% Magnesium stearate.

In an alternative embodiment of the second tablet the modified release portion consists essentially of:
(a) 80-90% Guaifenesin;
(b) 3-6% Dextromethorphan HBr;
(c) up to 5% Hypromellose;
(d) up to 2% Hydroxy ethylcellulose;
(e) up to 5% Microcrystalline cellulose;
(f) up to 1% Magnesium stearate.

In an alternative embodiment of the second tablet the modified release portion consists essentially of:
(a) 80-90% Guaifenesin;
(b) 3-6% Dextromethorphan HBr;
(c) up to 5% Hypromellose;
(d) up to 5% Hydroxyethylcellulose;
(e) up to 5% Microcrystalline cellulose; and
(f) up to 1% Magnesium stearate.

In an alternative embodiment of the second tablet the modified release portion consists essentially of:
(a) 80-90% Guaifenesin;
(b) 3-6% Dextromethorphan HBr;
(c) 5-10% Hypromellose;
(d) up to 5% Hydroxyethylcellulose;
(e) up to 5% Microcrystalline cellulose;
(f) up to 1% Magnesium stearate.

In an alternative embodiment of the second tablet the modified release portion consists essentially of:
(a) 85-90% Guaifenesin;
(b) 4-5% Dextromethorphan or a pharmaceutically acceptable salt thereof;
(c) 3-6% Hypromellose;
(d) 1-3% Carbomer;
(e) 0.5-1% Magnesium stearate.

In an alternative embodiment of the second tablet the modified immediate release portion consists essentially of:
(a) 90-93% Guaifenesin;
(b) 4-6% Dextromethorphan or a pharmaceutically acceptable salt thereof;
(c) 1-3% Carbomer;
(d) 0.5-1% Magnesium stearate.

For the avoidance of doubt the present disclosure covers all possible combinations and alternative embodiments of the first tablet and the immediate and modified release portions of the second tablet described herein so that the embodiments of the first tablet and the immediate and modified release portions of the second tablet can be used in any suitable combination which achieves the desired release profile of the active pharmaceutical ingredients.

The at least two tablets may comprise at least two different active pharmaceutical ingredients. For example, at least one of the at least two different active pharmaceutical ingredients may be present in only one of the compositions. Alternatively or cumulatively, at least one of the at least two different active pharmaceutical ingredients may be present in at least two of the compositions. For example, one of the at least two tablets may be an immediate release composition and the other one may be a controlled release composition, or both of the at least two compositions may be two different controlled release compositions for the benefits of delivering long lasting therapeutic benefits.

The at least two tablets may comprise at least two different active pharmaceutical ingredients, each of which may be comprised in only one composition or in more than one composition.

The term “controlled release composition” as used herein and in the appended claims refers to any composition that is not an immediate release composition, i.e., does not release all of the active pharmaceutical ingredients contained therein within a relatively short time (for example, within less than 1 hour, e.g., less than 0.5 hours, following ingestion of the capsule). Accordingly, this term is a generic term which encompasses, e.g., sustained (extended) release compositions, pulsed release compositions, delayed release compositions, and the like. Preferably, the controlled release compositions for use in the present invention release the one or more active ingredients contained therein continuously or intermittently over an extended period of time such as, e.g., at least about 2 hours, at least about 3 hours, at least about 4 hours, at least about 6 hours, or 12 hours. The desirable length of the time period of continuous or intermittent (e.g., pulsed) release depends, inter alia, on the plasma half-life of the active pharmaceutical ingredient and/or an active metabolite thereof. By way of non-limiting example, a capsule of the present invention may comprise a pharmaceutical composition comprising an active pharmaceutical ingredient about 70-100% of which is released upon contact of the composition with water over a period of 45 minutes. A second composition may release the same or a different active ingredient in a controlled manner over a predetermined time. Such a composition would comprise at least one active pharmaceutical ingredient, of which at least 80% would be released over a period of about 12 hrs. For example, the active ingredients may be formulated by using one or more swellable excipients which gel in the presence of water to achieve predetermined release characteristics.

The capsule of the present invention, the capsule may comprise a therapeutically effective amount of each active pharmaceutical ingredient contained therein and preferably is capable of providing a plasma concentration within the therapeutic range of each active pharmaceutical ingredient contained therein. The term “therapeutic range” as used herein refers to the range of active pharmaceutical ingredient levels (including active metabolite levels) within which most patients will experience a significant therapeutic benefits (including alleviation of symptoms) without an undesirable degree of adverse reactions.

The present invention also provides a gelatin or HPMC capsule for oral administration to a human subject which comprises at least two (and preferably only two) different pharmaceutical compositions which have been obtained by granulation of a mixture of one or more (e.g., one, two, three, four or five) active pharmaceutical ingredients and one or more excipients (e.g., one, two, three, four, five or six). At least one of the compositions is an immediate release composition and comprises at least one first active pharmaceutical ingredient and at least one of the compositions is a controlled release composition and comprises at least one second active ingredient which is the same as or different from the first active pharmaceutical ingredient. Further, the capsule comprises a therapeutically effective amount of the or each active ingredient contained therein.

According to the present invention care has to be taken that the bulkiness of the employed excipients as a whole is low enough to make it possible to incorporate at least two tablets different granular pharmaceutical compositions (each of which comprising at least one active pharmaceutical ingredient) into a capsule of a sufficiently small size for oral ingestion by a human subject. This does not exclude the possibility of including (preferably small amounts of bulky excipients into the compositions, as long as the overall dimensions of the tablets is still low enough to permit incorporation of at least two tablets in a single capsule that can be orally ingested by a human subject.

Modified release polymers that can be used in the compositions of the present invention include Acacia, Adipic Acid, Agar, Alginic Acid, Aliphatic Polyesters, Calcium Alginate, Carbomer, Carrageenan, Castor Oil, Cellaburate, Cellulose Acetate, Ceratonia, Colophony, Copovidone, Glyceryl Behenate, Glyceryl Monooleate, Glyceryl Monostearate, Glyceryl Palmitostearate, Hydroxypropyl Betadex, Hydroxypropyl Cellulose, Hydroxyethyl Cellulose, Hypromellose, Hypromellose Acetate Succinate, Methylcellulose, Polacrilin Potassium, Polycarbophil, Polydextrose, Polymethacrylates, Polyoxylglycerides, Polyvinyl Acetate Dispersion, Shellac, Sodium Alginate, Sodium Hyaluronate, Modified Starch, Sucrose Stearate, Microcrystalline Wax, White Wax, Yellow Wax, Xanthan Gum, Zein.

Hydrophilic polymers suitable for use in the sustained release portion include: one or more natural or partially or totally synthetic hydrophilic gums such as acacia, gum tragacanth, locust bean gum, guar gum, or karaya gum, modified cellulosic substances such as methylcellulose, hydroxymethylcellulose, hydroxypropyl methylcellulose, hydroxyethyl cellulose, hydroxypropyl cellulose, hydroxyethylcellulose, carboxyethylcellulose; proteinaceous substances such as agar, pectin, carrageen, and alginates; and other hydrophilic polymers such as carboxypolymethylene, gelatin, casein, zein, bentonite, magnesium aluminium silicate, polysaccharides, modified starch derivatives, and other hydrophilic polymers known to those of skill in the art or a combination of such polymers.

These hydrophilic polymers gel and dissolve slowly in aqueous acidic media thereby allowing the guaifenesin to diffuse from the gel in the stomach. When the gel reaches the intestines, it dissolves in controlled quantities in the higher pH medium, where the guaifenesin itself is fairly absorbable, to allow sustained release of guaifenesin throughout the digestive tract. Preferred hydrophilic polymers are the hydroxypropyl methylcelluloses such as those manufactured by The Dow Chemical Company and known as METHOCEL ethers. In one preferred embodiment of a sustained release formulation the hydrophilic polymer is a METHOCEL ether known as METHOCEL E10M or K100M.

Water-insoluble polymers which arc suitable for use in the sustained release portion are polymers which generally do not dissolve in solutions of a pH below 5, and dissolve more slowly in basic solutions than the hydrophilic polymer. Because the polymer is insoluble in low pH environments such as those found in gastric fluid, it aids in retarding drug release in those regions. Likewise, because the polymer dissolves more slowly in solutions of higher pH than hydrophilic polymers, it aids in retarding drug release throughout the intestines. This overall delayed release results in a more uniform serum concentration of guaifenesin.

The water-insoluble polymers suitable for use in the sustained release portion include: polyacrylic acids, acrylic resins, acrylic latex dispersions, cellulose acetate phthalate, polyvinyl acetate phthalate, hydroxypropyl methylcellulose phthalate, carbomer and other polymers common to those of skill in the art. In a preferred embodiment, a sustained release formulation comprises the acrylic resin CARBOPOL 974P supplied by BF Goodrich.

The sustained release portion of the present invention may further comprise pharmaceutical additives including, but not limited to: lubricants such as magnesium stearate, calcium stearate, zinc stearate, powdered stearic acid, hydrogenated vegetable oils, talc, polyethylene glycol, and mineral oil; colorants such as Emerald Green Lake and various FD&C colors; binders such as sucrose, lactose, gelatin, starch paste, acacia, tragacanth, povidone polyethylene glycol, Pullulan and corn syrup; glidants such as colloidal silicon dioxide and talc; surface active agents such as sodium lauryl sulfate, dioctyl sodium sulfosuccinate, tricthanolamine, polyoxyethylene sorbitan, poloxalkol, and quarternary ammonium salts; preservatives and stabilizers; excipients such as lactose, mannitol, glucose, fructose, xylose, galactose, sucrose, maltose, xylitol, sorbitol, chloride, sulfate and phosphate salts of potassium, sodium, and magnesium; and/or any other pharmaceutical additives known to those of skill in the art.

Colorants include, but are not limited to, Emerald Green Lake, FD&C Red #40, FD&C Yellow #6, FD&C Yellow #10, or FD&C Blue #1. In one preferred embodiment, a sustained release portion further comprises magnesium stearate and Emerald Green Lake. In another preferred embodiment, a sustained release formulation further comprises magnesium stearate and FD&C Blue #1 Aluminium Lake Dye.

The immediate release portion may comprise guaifenesin and various pharmaceutical additives such as disintegrants, lubricants, colorants, binders, glidants, surface active agents, preservatives, stabilizers, as described above and/or any other pharmaceutical additives known to those of skill in the art. Examples of suitable lubricant are as follows: Calcium Stearate, Glyceryl Behenate, Leucine, Magnesium Stearate, Mineral Oil, Myristic Acid, Palm Oil, Palmitic Acid, Poloxamer, Polyethylene Glycol, Potassium Benzoate, Sodium Benzoate, Sodium Lauryl Sulfate, Sodium Stearate, Sodium Stearyl Fumarate, Stearic Acid, Sucrose Stearate, Talc, Vegetable Oil, Zinc Stearate. Examples of suitable disintegrants are as follows: Carboxymethylcellulose Calcium, Carboxymethylcellulose Sodium, Sodium Lauryl Sulphate, Sodium Bicarbonate, Chitosan, Coilloidal Sillicon Dioxide, Croscarmellose Sodium, Crospovidone, Glycine, Guar Gum, Lactose, Magnesium Aluminum Silicate, Polacrilin Potassium, Povidone, Sodium Alginate, Sodium Starch Glycolate. Examples of suitable diluents are as follows: Calcium Carbonate, Calcium Lactate, Calcium Phosphate, Calcium Silicate, Calcium Sulfate, Cellaburate, Cellulose Acetate, Microcrystalline Cellulose, Silicified Microcrystalline Cellulose, Corn Syrup Solids, Dextrates, Dextrin, Dextrose, Erythritol, Ethylcellulose, Fructose, Inulin, Isomalt, Kaolin, Lactitol, Lactose, Magnesium Carbonate, Magnesium Oxide, Maltitol, Maltodextrin, Maltose, Mannitol, Triglycerides, Polydextrose, Simethicone, Sodium Bicarbonate, Sodium Carbonate, Sodium Chloride, Sorbitol, Sucrose, Sugar, Sulfobutylether β-Cyclodextrin, Sunflower Oil, Talc, Trehalose, Xylitol. Examples of suitable binders are as follows: Attapulgite, Calcium Carbonate, Calcium Lactate, Ceratonia, Colophony, Copovidone, Ethylcellulose, Ethylene Glycol and Vinyl Alcohol Grafted Copolymer, Gelatin, Glucose, Hydroxethylmethyl Celluose, Magnesium Aluminium Silicate, Methylcellulose, Polycarbophil, Polydextrose, Polyethylene Oxide, Polymethacrylates, Povidone, Pullulan, Vitamin E Polyethylene Glycol Succinate.

For the avoidance of any doubt, reference to a pharmaceutically active compound includes all enantiomers and stereoisomers thereof, and also all pharmaceutically acceptable salts or esters thereof. For example, naproxen includes naproxen sodium, pseudoephedrine includes pseudoephedrine hydrochloride, dextromethorphan includes dextromethorphan hydrobromide.

Embodiments of the invention will now be described by way of example only, with reference to the accompanying Figures in which:

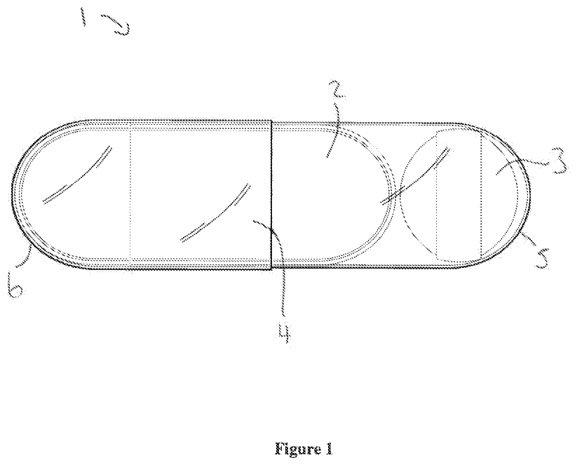

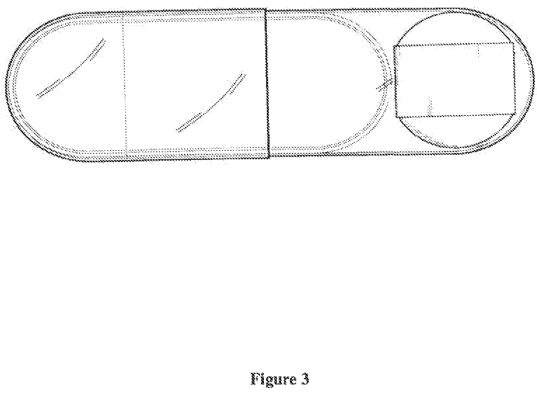

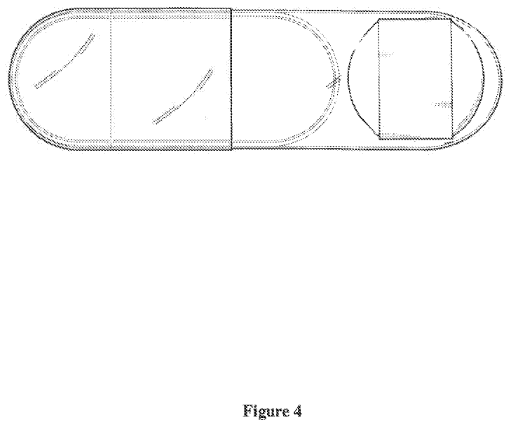

FIGS. 1-4 illustrate example embodiments of a pharmaceutical dosage form in accordance with the present invention;

### EXAMPLES

Referring firstly to FIG. 1, a pharmaceutical dosage form in accordance with the present invention is generally depicted at 1. The dosage form 1 comprises a first tablet 2 and a second tablet 3. The tablets 2 and 3 are encased in a gelatin or HPMC capsule 4. The gelatin or HPMC capsule comprises two separate shells 5 and 6. The capsule shells 5 and 6 have different dimensions such that one of the shells is larger than the other. Each of the shells 5 and 6 are provided with means in the form of grooves that allow the shells to reversibly engage with each other.

The pharmaceutical dosage form 1 is made by inserting the tablets into the longer shell 5. Shell 6 is then connected to shell 5 to form the final dosage form 1.

The tablets of the example embodiments of the present invention can be used using standard tableting procedures well-known to the man skilled in the art.

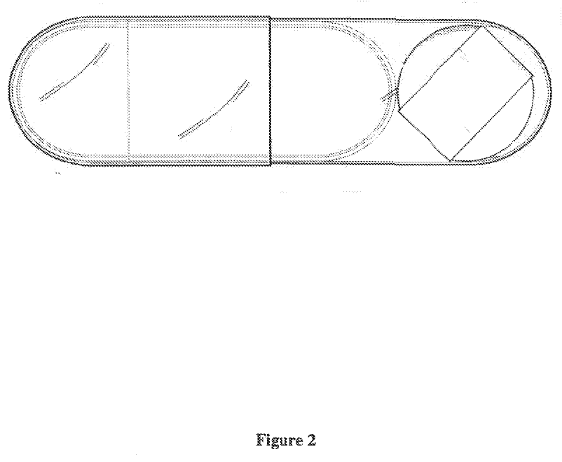

FIGS. 2-4 illustrate alternative embodiments of the present invention in which the tablets are different.

Example embodiments of the individual pharmaceutical compositions that can be used in the dosage form of the present invention will now be described by way of example only.

### Example 1

Tablet 1: Naproxen

| | | |
|---|---|---|
| Ingredient | mg/tablet | % Weight |

| | | | |
|---|---|---|---|
| Naproxen Sodium | 110.0 | mg | 73.33% |
| Microcrystalline Cellulose | 34.45 | mg | 22.96% |
| Povidone | 4.50 | mg | 3.0% |
| Mg Stearate | 1.05 | mg | 0.7% |
| Total Tablet | 150.0 | mg | 100.0% |

Tablet 2: Immediate/Sustained Release
Dextromethorphan and Guaifenesin

| | | |
|---|---|---|
| Ingredient | mg/tablet | % Weight |

| | | | |
|---|---|---|---|
| Guaifenesin | 600.0 | mg | 76.41% |
| Hypromellose | 50.00 | mg | 6.37% |
| MCC | 87.52 | mg | 11.15% |
| Dextromethorphan HBr | 30.0 | mg | 3.82% |
| Carbomer | 7.50 | mg | 0.96% |
| Sodium Starch Glycolate | 3.98 | mg | 0.51% |
| Colourant | 0.20 | mg | 0.025% |
| Mg Stearate | 6.0 | mg | 0.76% |
| Total Tablet | 785.2 | mg | 100.0% |

### Example 2

Tablet 1: Naproxen

| | | |
|---|---|---|
| Ingredient | mg/tablet | % Weight |

| | | | |
|---|---|---|---|
| Naproxen Sodium | 110.0 | mg | 51.16% |
| Microcrystalline Cellulose | 81.00 | mg | 37.67% |
| Crospovidone | 7.50 | mg | 3.49% |
| Croscarmellose sodium | 15.00 | mg | 6.98% |
| Mg Stearate | 1.50 | mg | 0.7% |
| Total Tablet | 215.0 | mg | 100.0% |

Tablet 2: Immediate/Sustained Release
Dextromethorphan and Guaifenesin

| | | |
|---|---|---|
| Ingredient | mg/tablet | % Weight |

| | | | |
|---|---|---|---|
| Guaifenesin | 600.0 | mg | 74.77% |
| Hypromellose | 19.00 | mg | 2.37% |
| MCC | 129.40 | mg | 16.12% |
| Dextromethorphan HBr | 30.0 | mg | 3.74% |
| Povidone | 7.00 | mg | 0.87% |
| Croscarmellose Sodium | 6.00 | mg | 0.74% |
| Hydroxy ethyl cellulose | 9.00 | mg | 1.12% |
| Colourant | 0.20 | mg | 0.025% |
| Mg Stearate | 1.9 | mg | 0.24% |
| Total Layer | 802.5 | mg | 100.0% |

### Example 3

Tablet 1: Naproxen

| | | |
|---|---|---|
| Ingredient | mg/tablet | % Weight |

| | | | |
|---|---|---|---|
| Naproxen Sodium | 110.0 | mg | 73.33% |
| Microcrystalline Cellulose | 14.55 | mg | 9.7% |
| Lactose | 12.65 | mg | 8.4% |
| Povidone | 4.65 | mg | 3.1% |
| Croscarmellose sodium | 7.15 | mg | 4.8% |
| Mg Stearate | 1 | mg | 0.7% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 200 | mg | 63.77% |
| Dextromethorphan HBr | 10 | mg | 3.19% |
| Microcrystalline Cellulose | 85 | mg | 27.10% |
| Povidone | 10 | mg | 3.19% |
| Croscarmellose Sodium | 8.5 | mg | 2.71% |
| Mg Stearate | 0.15 | mg | 0.05% |
| Total Layer | 313.65 | mg | 100.00% |

Modified Release Layer

| | | | |
|---|---|---|---|
| Guaifenesin | 400 | mg | 84.54% |
| Dextromethorphan HBr | 20 | mg | 4.23% |
| Hypromellose (K100M) | 24.5 | mg | 5.18% |
| Hydroxyethylcellulose | 12.25 | mg | 2.59% |
| Microcrystalline Cellulose | 14.75 | mg | 3.12% |
| Colourant | 0.1 | mg | 0.02% |
| Mg Stearate | 1.55 | mg | 0.33% |
| Total Layer | 473.15 | mg | 100.00% |
| Total Tablet | 786.8 | mg | 100.0% |

### Example 4

Tablet 1: Naproxen

| | | |
|---|---|---|
| Ingredient | mg/tablet | % Weight |

| | | | |
|---|---|---|---|
| Naproxen Sodium | 110 | mg | 13.33% |
| Microcrystalline Cellulose | 6 | mg | 4.00% |
| Lactose | 5 | mg | 3.33% |
| Povidone | 3 | mg | 2.00% |
| Sodium lauryl sulfate | 25 | mg | 16.67% |
| Mg Stearate | 1 | mg | 0.67% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 200 | mg | 63.77% |
| Dextromethorphan HBr | 10 | mg | 3.19% |
| Microcrystalline Cellulose | 85 | mg | 27.10% |
| Povidone | 10 | mg | 3.19% |
| Croscarmellose Sodium | 8.5 | mg | 2.71% |
| Mg Stearate | 0.15 | mg | 0.05% |
| Total Layer | 313.65 | mg | 100.00% |

Modified Release Layer

| | | | |
|---|---|---|---|
| Guaifenesin | 400 | mg | 84.54% |
| Dextromethorphan HBr | 20 | mg | 4.23% |
| Hypromellose (K100M) | 24.5 | mg | 5.18% |
| Hydroxyethylcellulose | 12.25 | mg | 2.59% |
| Microcrystalline Cellulose | 14.75 | mg | 3.12% |
| Colourant | 0.1 | mg | 0.02% |
| Mg Stearate | 1.55 | mg | 0.33% |
| Total Layer | 473.15 | mg | 100.00% |
| Total Tablet | 786.8 | mg | 100.0% |

### Example 5

Tablet 1: Naproxen

| | | |
|---|---|---|
| Ingredient | mg/tablet | % Weight |

| | | | |
|---|---|---|---|
| Naproxen Sodium | 110.0 | mg | 73.33% |
| Microcrystalline Cellulose | 14.55 | mg | 9.7% |
| Lactose | 12.65 | mg | 8.4% |
| Povidone | 4.65 | mg | 3.1% |
| Croscarmellose sodium | 7.15 | mg | 4.8% |
| Mg Stearate | 1 | mg | 0.7% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 130 | mg | 54.83% |
| Dextromethorphan HBr | 7.00 | mg | 2.95% |
| Microcrystalline Cellulose | 85.00 | mg | 35.85% |
| Povidone | 8.00 | mg | 3.37% |
| Croscarmellose Sodium | 7.00 | mg | 2.95% |
| Mg Stearate | 0.10 | mg | 0.04% |
| Total Layer | 237.10 | mg | 100.00% |

Modified Release Layer

| | | | |
|---|---|---|---|
| Guaifenesin | 470 | mg | 86.35% |
| Dextromethorphan HBr | 23 | mg | 4.23% |
| Hypromellose (K100M) | 17 | mg | 3.12% |
| Hydroxyethylcellulose | 17 | mg | 3.12% |
| Microcrystalline Cellulose | 15.5 | mg | 2.85% |
| Colourant | 0.1 | mg | 0.02% |
| Mg Stearate | 1.7 | mg | 0.31% |
| Total Layer | 544.3 | mg | 100.00% |
| Total Tablet | 781.40 | mg | 100.0% |

### Example 6

Tablet 1: Naproxen

| | | |
|---|---|---|
| Ingredient | mg/tablet | % Weight |

| | | | |
|---|---|---|---|
| Naproxen Sodium | 110 | mg | 73.33% |
| Microcrystalline Cellulose | 6 | mg | 4.00% |
| Lactose | 5 | mg | 3.33% |
| Povidone | 3 | mg | 2.00% |
| Sodium lauryl sulfate | 25 | mg | 16.67% |
| Mg Stearate | 1 | mg | 0.67% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 130 | mg | 54.83% |
| Dextromethorphan HBr | 7.00 | mg | 2.95% |
| Microcrystalline Cellulose | 85.00 | mg | 35.85% |
| Povidone | 8.00 | mg | 3.37% |
| Croscarmellose Sodium | 7.00 | mg | 2.95% |
| Mg Stearate | 0.10 | mg | 0.04% |
| Total Layer | 237.10 | mg | 100.00% |

Modified Release Layer

| | | | |
|---|---|---|---|
| Guaifenesin | 470 | mg | 86.35% |
| Dextromethorphan HBr | 23 | mg | 4.23% |
| Hypromellose (K100M) | 17 | mg | 3.12% |
| Hydroxyethylcellulose | 17 | mg | 3.12% |
| Microcrystalline Cellulose | 15.5 | mg | 2.85% |
| Colourant | 0.1 | mg | 0.02% |
| Mg Stearate | 1.7 | mg | 0.31% |
| Total Layer | 544.3 | mg | 100.00% |
| Total Tablet | 781.40 | mg | 100.0% |

### Example 7

Tablet 1: Naproxen

| | | |
|---|---|---|
| Ingredient | mg/tablet | % Weight |

| | | | |
|---|---|---|---|
| Naproxen Sodium | 110 | mg | 73.33% |
| Microcrystalline Cellulose | 34.45 | mg | 22.97% |
| Povidone | 4.5 | mg | 3.00% |
| Mg Stearate | 1.05 | mg | 0.7% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 85.5 | mg | 47.50% |
| Dextromethorphan HBr | 6.75 | mg | 3.75% |
| Microcrystalline Cellulose | 78.77 | mg | 43.76% |
| Hypromellose | 4.50 | mg | 2.50% |
| Sodium Starch Glycolate | 3.58 | mg | 1.99% |
| Mg Stearate | 0.90 | mg | 0.50% |
| Total Layer | 180.00 | mg | 100.00% |

Modified Release Layer

| | | | |
|---|---|---|---|
| Guaifenesin | 514.50 | mg | 88.83% |
| Dextromethorphan HBr | 23.25 | mg | 4.03% |
| Hypromellose | 27.50 | mg | 4.75% |
| Carbomer | 8.25 | mg | 1.42% |
| Colourant | 0.22 | mg | 0.04% |
| Mg Stearate | 5.50 | mg | 0.95% |
| Total Layer | 579.22 | mg | 100.00% |
| Total Tablet | 759.22 | mg | 100.0% |

### Example 8

Tablet 1: Naproxen

| | | |
|---|---|---|
| Ingredient | mg/tablet | % Weight |

| | | | |
|---|---|---|---|
| Naproxen Sodium | 110 | mg | 73.33% |
| Microcrystalline Cellulose | 34.45 | mg | 22.97% |
| Povidone | 4.5 | mg | 3.00% |
| Mg Stearate | 1.05 | mg | 0.7% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 76.00 | mg | 47.50% |
| Dextromethorphan HBr | 6.00 | mg | 3.75% |
| Microcrystalline Cellulose | 70.02 | mg | 43.76% |
| Hypromellose | 4.00 | mg | 2.50% |
| Sodium Starch Glycolate | 3.18 | mg | 1.99% |
| Mg Stearate | 0.80 | mg | 0.50% |
| Total Layer | 160.00 | mg | 100.00% |

Modified Release Layer

| | | | |
|---|---|---|---|
| Guaifenesin | 424.00 | mg | 91.74% |
| Dextromethorphan HBr | 24.00 | mg | 5.19% |
| Carbomer | 9.90 | mg | 2.14% |
| Colourant | 0.26 | mg | 0.06% |
| Mg Stearate | 4.00 | mg | 0.87% |
| Total Layer | 462.16 | mg | 100.00% |
| Total Tablet | 622.16 | mg | 100.0% |

### Example 9

Tablet 1: Naproxen

| | | |
|---|---|---|
| Ingredient | mg/tablet | % Weight |

| | | | |
|---|---|---|---|
| Naproxen Sodium | 110 | mg | 73.33% |
| Microcrystalline Cellulose | 14.5 | mg | 9.67% |
| Povidone | 4.75 | mg | 3.17% |
| Mg Stearate | 0.75 | mg | 0.7% |
| Lactose | 10.00 | mg | 6.67% |
| Croscarmellose Sodium | 10.00 | mg | 6.67% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 85.5 | mg | 47.50% |
| Dextromethorphan HBr | 6.75 | mg | 3.75% |
| Microcrystalline Cellulose | 78.77 | mg | 43.76% |
| Hypromellose | 4.50 | mg | 2.50% |
| Sodium Starch Glycolate | 3.58 | mg | 1.99% |
| Mg Stearate | 0.90 | mg | 0.50% |
| Total Layer | 180.00 | mg | 100.00% |

Modified Release Layer

| | | | |
|---|---|---|---|
| Guaifenesin | 514.50 | mg | 88.83% |
| Dextromethorphan HBr | 23.25 | mg | 4.03% |
| Hypromellose | 27.50 | mg | 4.75% |
| Carbomer | 8.25 | mg | 1.42% |
| Colourant | 0.22 | mg | 0.04% |
| Mg Stearate | 5.50 | mg | 0.95% |
| Total Layer | 579.22 | mg | 100.00% |
| Total Tablet | 759.22 | mg | 100.0% |

### Example 10

Tablet 1: Naproxen

| | | |
|---|---|---|
| Ingredient | mg/tablet | % Weight |

| | | | |
|---|---|---|---|
| Naproxen Sodium | 110 | mg | 73.33% |
| Microcrystalline Cellulose | 14.5 | mg | 9.67% |
| Povidone | 4.75 | mg | 3.17% |
| Mg Stearate | 0.75 | mg | 0.7% |
| Lactose | 10.00 | mg | 6.67% |
| Croscarmellose Sodium | 10.00 | mg | 6.67% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 76.00 | mg | 47.50% |
| Dextromethorphan HBr | 6.00 | mg | 3.75% |
| Microcrystalline Cellulose | 70.02 | mg | 43.76% |
| Hypromellose | 4.00 | mg | 2.50% |
| Sodium Starch Glycolate | 3.18 | mg | 1.99% |
| Mg Stearate | 0.80 | mg | 0.50% |
| Total Layer | 160.00 | mg | 100.00% |

Modified Release Layer

| | | | |
|---|---|---|---|
| Guaifenesin | 424.00 | mg | 91.74% |
| Dextromethorphan HBr | 24.00 | mg | 5.19% |
| Carbomer | 9.90 | mg | 2.14% |
| Colourant | 0.26 | mg | 0.06% |
| Mg Stearate | 4.00 | mg | 0.87% |
| Total Layer | 462.16 | mg | 100.00% |
| Total Tablet | 622.16 | mg | 100.0% |

### Example 11

Tablet 1: Naproxen

| | | |
|---|---|---|
| Ingredient | mg/tablet | % Weight |

| | | | |
|---|---|---|---|
| Naproxen Sodium | 110 | mg | 72.37% |
| Microcrystalline Cellulose | 9.25 | mg | 6.09% |
| Povidone | 4.75 | mg | 3.13% |
| Mg Stearate | 1.00 | mg | 0.66% |
| Sodium Lauryl Sulphate | 25.00 | mg | 16.45% |
| Croscarmellose Sodium | 2.00 | mg | 1.32% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 85.5 | mg | 47.50% |
| Dextromethorphan HBr | 6.75 | mg | 3.75% |
| Microcrystalline Cellulose | 78.77 | mg | 43.76% |
| Hypromellose | 4.50 | mg | 2.50% |
| Sodium Starch Glycolate | 3.58 | mg | 1.99% |
| Mg Stearate | 0.90 | mg | 0.50% |
| Total Layer | 180.00 | mg | 100.00% |

Modified Release Layer

| | | | |
|---|---|---|---|
| Guaifenesin | 514.50 | mg | 88.83% |
| Dextromethorphan HBr | 23.25 | mg | 4.03% |
| Hypromellose | 27.50 | mg | 4.75% |
| Carbomer | 8.25 | mg | 1.42% |
| Colourant | 0.22 | mg | 0.04% |
| Mg Stearate | 5.50 | mg | 0.95% |
| Total Layer | 579.22 | mg | 100.00% |
| Total Tablet | 759.22 | mg | 100.0% |

### Example 12

Tablet 1: Naproxen

| | | |
|---|---|---|
| Ingredient | mg/tablet | % Weight |

| | | | |
|---|---|---|---|
| Naproxen Sodium | 110 | mg | 72.37% |
| Microcrystalline Cellulose | 9.25 | mg | 6.09% |
| Povidone | 4.75 | mg | 3.13% |
| Mg Stearate | 1.00 | mg | 0.66% |
| Sodium Lauryl Sulphate | 25.00 | mg | 16.45% |
| Croscarmellose Sodium | 2.00 | mg | 1.32% |
| Total Tablet | 150.0 | mg | 100.0% |

Tablet 2: Immediate/Modified Release
Dextromethorphan and Guaifenesin

| | | |
|---|---|---|
| Ingredient | mg/tablet | % Weight |

Immediate Release Layer

| | | | |
|---|---|---|---|
| Guaifenesin | 76.00 | mg | 47.50% |
| Dextromethorphan HBr | 6.00 | mg | 3.75% |
| Microcrystalline Cellulose | 70.02 | mg | 43.76% |
| Hypromellose | 4.00 | mg | 2.50% |
| Sodium Starch Glycolate | 3.18 | mg | 1.99% |
| Mg Stearate | 0.80 | mg | 0.50% |
| Total Layer | 160.00 | mg | 100.00% |

Modified Release Layer

| | | | |
|---|---|---|---|
| Guaifenesin | 424.00 | mg | 91.74% |
| Dextromethorphan HBr | 24.00 | mg | 5.19% |
| Carbomer | 9.90 | mg | 2.14% |
| Colourant | 0.26 | mg | 0.06% |
| Mg Stearate | 4.00 | mg | 0.87% |
| Total Layer | 462.16 | mg | 100.00% |
| Total Tablet | 622.16 | mg | 100.0% |

An advantage of the present invention is that there is provided a pharmaceutical dosage form which brings together two or more medicaments and which is simple to manufacture.

In addition, the pharmaceutical dosage form of the present invention allows for individual tablets having different dimensions to be combined into a single administrable dosage form.

The present application is primarily directed to the combination of two tablets. However, the person skilled in the art will easily be able to adapt the pharmaceutical dosage form to the presence of more than two tablets, especially three or four tablets.

In addition, it is possible by to incorporate separating sheets or walls into the capsule where necessary. For example, it will be possible for the person skilled in the art to envisage pharmaceutical dosage forms comprising multi-compartment gelatin or HPMC capsules. Such a gelatin or HPMC capsule therefore comprises at least two compartments each containing a tablet according to the invention, which are separated from one another by a separating wall.

Further modifications and developments can be made without departing from the scope of the invention described herein.

1. An oral pharmaceutical dosage form comprising a capsule containing at least two tablets, each tablet containing at least one different pharmaceutically active ingredient wherein each of the at least two tablets have different dimensions.

2. An oral pharmaceutical dosage form as claimed in claim 1, wherein after dissolution of the capsule the at least two tablets release the active pharmaceutical ingredients contained therein comparably to each of the at least two tablets being administered individually.

3. An oral pharmaceutical dosage form as claimed in claim 1, wherein the at least two tablets can have the same or a different geometric form, the same or a different weight, and the same or a different volume with the proviso that at least one of the geometric form, weight or volume is different between the tablets.

4. An oral pharmaceutical dosage form as claimed in claim 1, wherein the capsule has a length of up to 35 mm, a width of up to 15 mm and a depth of up to 15 mm.

5. An oral pharmaceutical dosage form as claimed in claim 4, wherein the capsule has a length of 25-30 mm, a width of 8-10, and a depth of 8-10 mm.

6. An oral pharmaceutical dosage form as claimed in claim wherein the capsule has a length of 20-24 mm, a width of 8-10, and a depth of 8-10 mm.

7. An oral pharmaceutical dosage form as claimed in claim 1, wherein the at least two different tablets may comprise one or more active pharmaceutical active ingredients selected from analgesics, antihistamines, decongestants, antitussives, expectorants, vitamins, probiotics, anti-inflammatories, anti-infectives, antibiotics, acid reducers, and laxatives.

8. An oral pharmaceutical dosage form as claimed in claim wherein the at least two tablets comprise an analgesic, an expectorant and at least one additional active pharmaceutical ingredient.

9. An oral pharmaceutical dosage form as claimed in claim wherein the analgesic is selected from naproxen, ketoprofen, diclofenac, ibuprofen, paracetamol, aspirin and flurbiprofen.

10. An oral pharmaceutical dosage form as claimed in claim wherein the expectorant can be guaifenesin or N-acetyl cysteine (NAC).

11. An oral pharmaceutical dosage form as claimed in claim wherein the at least one other drug can be selected from an antitussive or cough suppressant such as dextromethorphan, dextromethorphan hydrobromide, codeine, codeine phosphate, codeine sulphate, diphenhydramine citrate, diphenhydramine hydrochloride and diphenhydramine citrate, a decongestant such as phenylephrine hydrochloride, phenylpropanolamine hydrochloride, pseudoephedrine hydrochloride or ephedrine, an antihistamine such as chlorpheniramine maleate, brompheniramine maleate, phenindamine tartrate, pyrilamine maleate, doxylamine succinate, phenyltoloxamine citrate, diphenhydramine hydrochloride, diphenhydramine citrate, promethazine, and clemastine fumerate, or a combination thereof.

12. An oral pharmaceutical dosage form as claimed in claim 11, wherein the at least one other drug is dextromethorphan hydrobromide or pseudoephedrine hydrochloride.

13. An oral pharmaceutical dosage form as claimed in claim 1, wherein the first tablet is an immediate release tablet which comprises naproxen, and the second tablet comprises an immediate release portion comprising guaifenesin and dextromethorphan and a sustained release portion comprising guaifenesin and dextromethorphan.

14. (canceled)

15. An oral pharmaceutical dosage form as claimed in claim 13, wherein the first tablet comprises:
(a) 70-75 wt % Naproxen;
(b) 20-25 wt % Microcrystalline cellulose;
(c) up to 5 wt % Povidone; and
(d) up to 1 wt % Magnesium stearate.

16.-24. (canceled)

25. An oral pharmaceutical dosage form as claimed in claim 13, wherein the immediate release portion of the second tablet comprises:
(a) 40-50 wt % Guaifenesin;
(b) up to 5 wt % Dextromethorphan HBr;
(c) 40-50 wt % Microcrystalline cellulose;
(d) up to 5 wt % Hypromellose;
(e) up to 5 wt % Sodium starch glycolate; and
(f) up to 1 wt % Magnesium stearate.

26. (canceled)

27. (canceled)

28. An oral pharmaceutical dosage form as claimed in claim 13, wherein the immediate release portion of the second tablet comprises:
(a) 40-50 wt % Guaifenesin;
(b) up to 5 wt % Dextromethorphan or a pharmaceutically acceptable salt thereof;
(c) 40-50 wt % Microcrystalline cellulose;
(d) up to 5 wt % Hypromellose;
(e) up to 5 wt % Sodium starch glycolate; and
(f) up to 1 wt % Magnesium stearate.

29. (canceled)

30. An oral pharmaceutical dosage form as claimed in claim 13, wherein the modified release portion of the second tablet comprises:
(a) 80-90 wt % Guaifenesin;
(b) up to 5 wt % Dextromethorphan HBr;
(c) up to 10 wt % Hypromellose;
(d) up to 5 wt % Carbomer; and
(e) up to 1.55 wt % Magnesium stearate.

31.-33. (canceled)

34. An oral pharmaceutical dosage form as claimed in claim 13, wherein the modified release portion of the second tablet comprises:
(a) 85-90 wt % Guaifenesin;
(b) 4-5 wt % Dextromethorphan or a pharmaceutically acceptable salt thereof;
(c) 3-6 wt % Hypromellose;
(d) 1-3 wt % Carbomer; and
(e) 0.5-1 wt % Magnesium stearate.

35. An oral pharmaceutical dosage form as claimed in claim 13, wherein the modified release portion of the second tablet comprises:
(a) 90-93 wt % Guaifenesin;
(b) 4-6 wt % Dextromethorphan or a pharmaceutically acceptable salt thereof;
(c) 1-3 wt % Carbomer; and
(d) 0.5-1 wt % Magnesium stearate.

36. (canceled)

37. A method of treatment of symptoms of cough, cold, and/or flu comprising administering the oral pharmaceutical dosage form of claim 1 to a patient in need thereof, wherein a single dose of the oral pharmaceutical dosage form provides a therapeutic effect for a period of up to 12 hours.
